(19) United States
(12) Patent Application Publication
(10) Pub. No.: US 2013/0216972 A1
(43) Pub. Date: Aug. 22, 2013

(54) PROCESS AND APPARATUS FOR COMPUTER-CONTROLLED PREPARATION OF TEETH

(71) Applicant: Laszlo Kolozsvary, Piliscsaba (HU)

(72) Inventor: Laszlo Kolozsvary, Piliscsaba (HU)

(21) Appl. No.: 13/825,130

(22) PCT Filed: Jul. 15, 2011
(86) PCT No.: PCT/HU11/00073

§ 371 Date: Mar. 20, 2013

(30) Foreign Application Priority Data
Jul. 22, 2010 (HU) P1000387

(51) Int. Cl. A61C 13/00; A61C 19/00; A61B 1/24; A61C 1/00; A61C 5/00

(52) U.S. Cl. CPC A61C 13/0004; A61C 1/0046; A61C 1/0069; A61C 5/007; A61B 1/24; A61C 19/00
USPC 433/29; 433/219

(57) ABSTRACT

The subject of the invention is automation of process and apparatus for computer-controlled preparation of teeth, during said process preparing the tooth-stump(s) holding the prosthetic piece as pillar(s), takes place on basis of a digital plan of three dimensions, with a combined laser head of small size, matching the size of the oral cavity, moving along a trajectory fixed to a frame of face-bow controlled by a computer, preparing teeth-stump(s) before placing into the mouth the prosthetic piece produced by CAD/CAM technique.

The subject of the invention is automation of process and apparatus for computer-controlled preparation of teeth, during said process preparing the tooth-stump(s) holding the prothetic piece as pillar(s), takes place on basis of a digital plan of three dimensions, with a combined laser head of small size, matching the size of the oral cavity, moving along a trajectory fixed to a frame of face-bow controlled by a computer, preparing teeth-stump(s) before placing into the mouth the prothetic piece produced by CAD/CAM technique—bridge, crown, insert, etc.

Dentistry as a profession can be credited with significant improvement during the last ten years both regarding diagnostics and prosthodontics resulting from the application of digital technologies.

Due to the use of 3-dimensional pictures X-ray recordings could be made of the appropriate part of the patient's maxillo-mandibular area in space, and this technique is available for a wide range of patients resulting from the gradual decrease of the price of the machines. CAD/CAM technique has revolutionized prosthodontics. Between these well-developed technological steps stands the dentist with their “shaking hands” and relatively low optical definition ability.

The CAD/CAM technique becoming more and more widespread in prosthodontics makes possible a realization of the prosthesis with the accuracy of a hundredth millimeter. The dentist is not able to produce such high accuracy when working manually. Obviously there is a difference between dentists regarding manual skills On top of that, depending on the individual emotional and fitness conditions, even dentists of good manual skills have bad days. Under difficult anatomical conditions preparation of the stump might take place blind. Under such conditions even in case of a laboratory working very accurately there is a risk of inappropriate shape of the ready denture—precisely because of great accuracy—resulting in the impossibility of placing it into the oral cavity. This case a subsequent adjustment is needed, which on the other hand ruins the precise work.

Nowadays there are modern devices for preparational works, allowing quite accurate preparation, for example laser heads working on hard textures—at present teeth. One of the most modern laser milling machines is the erbium laser provided with exchangable sapphire photo-conductor, as device milling teeth as hard texture. The size of these laser heads is quite small, and they are apt to be used in the oral cavity.

According to the state of the art known the Cone Beam CT as a method in prosthodontics. This equipment produces a 3D digital impression for the dentist, which can be manipulated by revolting it into every direction of the space, and different measuring can be performed on it. The system produces a digital database in DICOM format, that can be manipulated as a matrix, and digital designing can be carried out. On this digital matrix the dentist plans the course and form of stump preparation. This plan has dual aim. On the one hand it will be the final version of the plan for the automated milling device, on the other hand the digital matrix can be made compatible with the CAD/CAM systems currently used in prosthetic dentistry, by this way becoming the digital pattern of the prosthodontics work eliminating the inaccuracy of impressions and mold making.

Use of different maxillo-mandibular areas is known for making prostheses to determine exact positions.

The WO 001 0482 patent description makes known a state of the art process for the restoration of at least one tooth, or for the restoration of teeth of one area of the jaw, by using computer-ontrolled CAD/CAM technology, primarily in case of adjoining molars or incisors with great accuracy.

The WO 02 089 695 patent description makes known a process for digital impression taking, during which the prosthetic device is made on basis of a X-ray recording.

The DE 1951 8702 patent publication makes known a process and device for the computer-assisted restoration of teeth. In a first stage of the process the geometry of the tooth to be restored is measured with the aid of a measuring device and electronically stored. In a second stage the tooth image is interpreted, in a third stage the restoring element is constructed using CAD. In a fourth stage the restoring element is ground from a block of suitable material with the aid of a numerically controlled grinder (CAM). The CAD/CAM operation is transferred to a powerful central computer having the appropriate tools. The computer operates automatically to the greatest possible extent; where necessary it is run by an experienced operator and is connected via a data line to the 3D measuring device and the grinder.

The CN101450010 patent publication makes known an oral cavity restoring body making equipment based on CAD/CAM technology. The invention discloses a digitalization oral cavity restoring body making equipment comprising a laser three-dimensional scanning system for acquiring tooth jaw model and articulation recording three-dimensional data, an oral cavity restoring CAD/CAM design system for rapidly making the oral cavity restoring body and a specific digital control equipment for machining oral cavity restoring body. The laser three-dimensional scanning system is communicated with the oral cavity restoring CAD/CAM design system, the oral cavity restoring CAD /CAM design system is communicated with the specific digital control equipment. The invention has simple operation, high efficiency and manufacturing accuracy, can obviously shorten the curing period, improve the work efficiency, alleviate the patient pain, simultaneously improve the quality of the oral cavity restoring body

In case of the former three solutions the dentist “submits” a ready tooth-stump to the machine, which is scanned by the machine and a digital plan is made on basis of it, and the prosthetic device is milled automatically from a given block by the machine.

The DE 1992 2870 patent publication makes known automatic adjustment method for the color, translucence, brightness and fluorescence of dental restoration work. The method comprises submitting a set of CAD data about the restoration work to an iterative process comprising: (a) analyzing these properties for a natural tooth or denture; (b) calculating the required values for the restoration; (c) applying material to the structure of the restoration work to produce the required shape and properties; with (d) correcting iterations until the required properties have been attained.

During the process a digital spatial model is made from a digital database, which is needed for the scanning in the oral cavity.

The US 2008 131 833 patent publication makes known a surface mapping and generating device which has devices for the process and/or cost optimization, in particular raw material recovery devices, an automatic control of the intensity of a used laser light, devices for carrying out a calibrating procedure by evaluating overlap errors at matching points, devices for archiving especially three-dimensional data of the jaw and/or for modeling the bite position of the upper and the lower jaws, devices for the optimized preparation of at least one dental stump for the production and placement of a dental prosthesis thereon and/or devices for taking into consideration the bite position of the upper and lower jaws.

In the state of the art is the tooth-stump preparation with hard laser beam is furthermore known, and the laser device performing it as well. EP 053 0574, EP 052 3506, EP 056 2988, U.S. Pat. No. 5,388,988, U.S. Pat. No. 5,474,449, U.S. Pat. No. 5,401,171 and WO 98 30168 patent descriptions make known laser devices making tooth-stump laser preparations.

JP 6000 158 and U.S. Pat. No. 5,051,823 patent descriptions make known laser heads provided with video camera as control optics used in prosthetic dentistry.

FR 277 3619 patent description makes known air-intake tubes and water-intake tubes on the same place as the laser unit for the cooling of the appropriate part of the mouth.

The JP5309101 patent publication makes known a dental treatment device to carry out the treatment process, the positioning, and the form measuring by a single machine, and to carry out the processing while measuring. A multijoint holding handle which can control the positioning freely is provided to a stand, a laser oscillator held to the holding handle and to radiate lasers, or an optical fiber to lead and guide from the laser oscillator is provided to the holding handle, and a measuring device to measure the three-dimensional form by utilizing the reflection beams by the radiating lasers is provided, so as to form this dental treatment device. The treatment and the measurement are carried out concurrently by the radiating lasers. During the treatment, a cooling and washing water is fed from a pump device through a hose, and the washing solution in the mouth is sucked and exhausted to an exhaust solution tank by a suction pump through a hose.

The drawback of the known solutions is, that every step of tooth restoration is automated, computer controlled, and very accurate, except for the preparation of the tooth stump, which is made manually by the dentist still to date. Modern devices, for example laser heads with control optics are helpful at the preparation of tooth stump, however difficulties resulting from complicated anatomical conditions, as well as subjective factors—individual skills, tiredness, trembling of hands, actual psychical condition of the dentist—can not be overcome.

When working out the solution according to the invention we aimed to develop a process and apparatus belonging to it, making possible the absolutely precise preparation of the tooth-stump on basis of a digital plan.

When creating the solution according to the invention we realized, that in case the planing of the preparation is accurately made by the dentist on basis of a 3D X-ray image on a computer with the help of a designing program made for this purpose, then on basis of the ready digital plan the prosthetic device is produced with CAD/CAM technology, with great accuracy, and during the preparation of the tooth-stump(s) the dentist makes the preparation of the pillar tooth (teeth) with computer control, also on basis of the ready digital plan, with a combined laser head of small size, matching the size of the modified oral cavity, moving along a trajectory fixed to a frame of modified face-bow controlled by a computer, and said combined laser head contains a hard laser miller, control laser optics and cooling openings, then the set aim can be achieved.

The invention is a process for the automation of dental stump preparation, during which process the designing of the preparation of the dental stump(s) serving as pillar tooth/teeth for the fixing of the prothetic piece—bridge, crown, insert,—to be placed into the oral cavity takes place by the dentist on basis of a three-dimensional X-ray recording, taken from the relative spot of the jaw, on computer with the help of a designing-program made for this purpose, then, on basis of the complete digital plan, producing the prosthesis takes place with CAD/CAM technology, then on basis of the same digital plan, preparation of the dental stump(s) serving as pillar tooth/teeth takes place also by the dentist with laser head dental milling device, which is characterized by that, during the preparation of the dental stump(s)

as Step 1 the dentist, or a professionally qualified person persuades the patient's mandible into maximum opening position with the help of the telescopic frame of maxillo-mandibular fixing and fixes it there,
as Step 2 on basis of the complete digital plan the computer directs the combined laser head in the car to the pillar tooth to be prepared,
as Step 3 the laser miller with mobility towards every direction of the space situated on the combined laser head directed to the proper spot, carries out the preparation of the pillar tooth on basis of the complete digital plan, digitally controlled by the computer, under the supervision of the dentist, or a professionally qualified person, with the supervision of the control optics.

In a preferred application of the process according to the invention at first the upper part of the frame of maxillo-mandibular fixing, from the nose bridge to the oral cavity is fixed to the patient's maxillo-mandibular area, then, following this the lower part of the frame of maxillo-mandibular fixing, the part from the oral cavity to the bottom of the jaw is fixed to the patient's maxillo-mandibular area with the help of the pneumatically functioning telescopic formation of the frame of maxillo-mandibular fixing conforming with the measurements of the given patient.

In another preferred application of the process according to the invention during preparation the control optics automatically finds the reference points in the digital frame, marked during the computer designing, and comparing with them, controls the laser miller, so the combined laser head is directed by the computer on the complete digital plan in such a way, that it compares the digital frame with the signals sent by the control optics.

In a further preferred application of the process according to the invention the laser miller and the control optics operate continously, parallel with each other.

In a further preferred application of the process according to the invention the laser miller and the control optics operate alternating, withing short, subsequent periods.

In a further preferred application of the process according to the invention during milling the cooling water coming from the cooling openings cools down continously the actual tooth under preparation.

The invention is further an apparatus for the automation of dental stump preparation, preferably for realizing the process according to the invention, the said apparatus is provided with control optics for surveying the preparation of tooth stump(s), which is made by a combined laser head containing a laser miller emitting hard laser beam, which is characterized by that,
the supporting, fixing points of the telescopic frame of maxillo-mandibular fixing on the patient's maxillo mandibular area comprising part of the automatic tooth stump preparing apparatus, are as follows:
   the nose ridge—with the help of the cushion,
   the acces of external acoustic meatus (5) of both ears (4)—with the help of a metallic or plastic ball (17) each,
   the palate (7)—with the help of the rubbered palate support (19) of ball shape,
   the area of retromolar gap (12) situated on both mandibles—with the help of the retromolar support (pad) (18),
   the part of the jaw tip (11) bending back,—with the help of the vacuum pad for fixing jaw tip (20) with polystyrene balls, bearing below the jaw tip (11),
the combined laser head (26) carrying out the milling travells in the car (25) placed on the horse-shoe-shaped guide rail (24) following the arc of the teeth, fixed on the frame of maxillo-mandibular fixing (14) of the automatic tooth stump preparing apparatus.

In one preferred realization of the apparatus according to the invention, the supporting points of the frame of maxillo-mandibular fixing, are connected with each other by a telescopic stiffening stud, movable pneumatically, this way the complete frame of maxillo-mandibular fixing is a uniform structure.

In another preferred realization of the apparatus according to the invention, each part of the frame of maxillo-mandibular fixing, is made of materials of easy cleansing and desinfecting, preferably from metal, plastic, or rubberized, or siliconized metal or plastic.

In a further preferred realization of the apparatus according to the invention, in case the pillar tooth to be prepared is in the upper toothrow, then the combined laser head situated in the car is on the upper part of the guide rail, and the guide rail of horse-shoe shape itself is set near the upper toothrow.

In a further preferred realization of the apparatus according to the invention, in case the pillar tooth to be prepared is in the lower toothrow, then the combined laser head in the car is situated on the lower part of the guide rail, and the guide rail of horse-shoe shape itself is located near the lower toothrow.

In a further preferred realization of the apparatus according to the invention, the control optics situated concentrically around the laser miller on the combined laser head and the cooling openings preferably four pieces of cooling openings are situated symmetrically outside the control optics.

In a further preferred realization of the apparatus according to the invention, the laser miller is preferably erbium laser miller.

In a further preferred realization of the apparatus according to the invention, the control optics of the combined laser head is preferably a laser control optics emitting soft laser beams.

In a further preferred realization of the apparatus according to the invention, the combined laser head in the car is positioned on a lever, and the computer controlling the preparation directs the movement of the lever, so the combined laser head is able to move along the hemisphere shell.

In a further preferred realization of the apparatus according to the invention, the combined laser head in the car, able to move along the hemisphere shell is located on the shell of a hemisphere, and the movement of the combined laser head on the shell of the hemisphere is directed by the computer controlling the preparation.

In a further preferred realization of the apparatus according to the invention, the following complements join the combined laser head:
glass fibre leading the hard laser beam from the laser generator to the laser miller,
another glass fibre leading the soft laser beam carrying out the control in the control optics to the computer controlling the milling,
tubes, preferably four pieces of tubes, leading the cooling water to the cooling openings.

In a further preferred realization of the apparatus according to the invention, additional supplementary devices of the automatic tooth stump preparing apparatus are as follows:
a silicon-rubber sheet usually used in dentistry, separating safely the oral cavity from the pharynx, fixed to the proper parts of the frame of maxillo-mandibular fixing at five points, and these five points are the following:
   two points at the retromolar gap,
   one point in the middle of the palate
   two points at the tuber maxilae on the upper jaw,
as well as a water suction tube (saliva pump) generally used in dentistry ensuring the removal of the cooling water and the saliva in a proper way.

The apparatus according to the invention is furthermore set forth by the enclosed drawings:

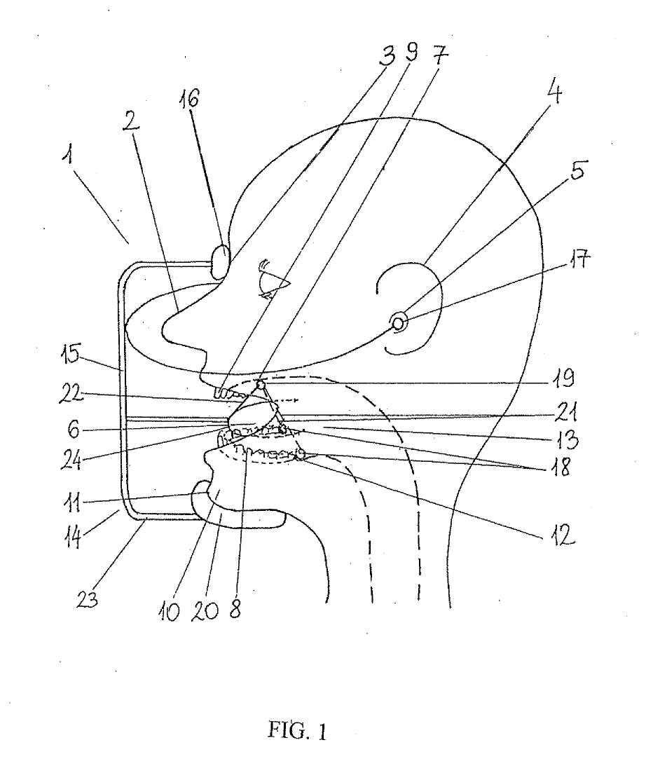

FIG. 1 shows the automatic apparatus for tooth-stump preparation in side view, on a schematical representation, fixed onto the maxillo-mandibular area 1 of the patient.

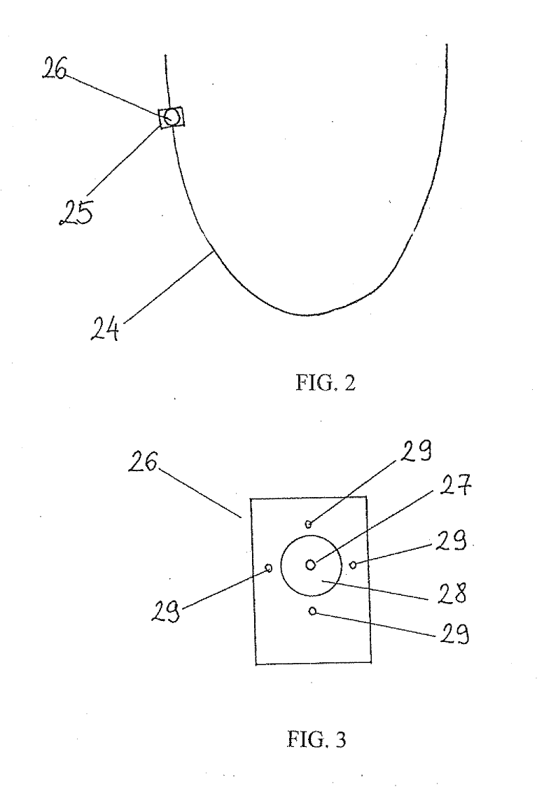

FIG. 2 shows a detail of the automatic apparatus for tooth-stump preparation introduced in FIG. 1 situated in the oral cavity 6 in top view.

FIG. 3 shows the combined laser head 26 in details.

FIG. 1 shows the automatic apparatus for tooth-stump preparation in side view, on a schematical representation, fixed onto the maxillo-mandibular area 1 of the patient. The frame of maxillo-mandibular fixing 14 with the guide rail 24 and the car 25 travelling on the guide rail 24 can be seen in the drawing. The upper part, that is the part from the nose ridge 3 to the oral cavity 6 of the frame of maxillo-mandibular fixing 14 creates the modified maxillo-mandibular area 15. The lower part of the frame of maxillo-mandibular fixing 14 is the part from the oral cavity 6 to the lower part of the jaw 10.

The frame of maxillo-mandibular fixing 14 bears up the maxillo-mandibular area 1 on the following points: with the help of the cushion 16 on the nose ridge 3 situated above the nose 2, at both ears 4 at the access of external acoustic meatus 5 with the help of a metallic or plastic ball 17 each, behind the lower toothrow 8, on the retromolar gap 12, both on the right- and left side with the help of a retromolar support (pad) 18 each, on the palate 7 with the help of the palate support 19, which is a rubbered ball, and at the jaw tip 11 of the jaw 10, with the help of the vacuum pad for fixing jaw tip 20 bending back to the jaw tip 11, fixing the jaw 10 from the bottom.

The frame of maxillo-mandibular fixing 14 creates a uniform frame system, each point of which is connected with the other points of the frame system. The supporting points of the frame system above the mouth, situated outside the oral cavity 6, namely the supporting point on the nose ridge 3 with the help of the cushion 16, and the supporting points at the two accesses of external acoustic meatus 5 with the help of a ball 17 each are connected with the modified maxillo-mandibular area 15. From among the supporting points of the frame system situated within the oral cavity 6, the supporting points of the two retromolar gaps 12 are connected with the help of the retromolar support (pad) 18 on both sides with the stud for sustaining jaw 21, the supporting point on the palate 7 is connected with the help of the palate support 19 with each other and with the stud for supporting palate 22 and the modified maxillo-mandibular area 15. The supporting point of the frame system below the mouth, outside the oral cavity 6, namely the supporting point on the jaw tip 11 fixing the jaw tip 11 from below with the help of the vacuum pad for fixing jaw tip 20 is connected with the stud for fixing jaw tip 23 to the frame of maxillo-mandibular fixing 14.

The frame of maxillo-mandibular fixing 14 is a uniform, but not fixed frame system, namely the studs connecting the supporting points are of telescopic structure, preferably the length of each stud can be adjusted in a pneumatic way, conforming with the measurements of the patient. The guide rail 24 of the frame of maxillo-mandibularfixing 14 fixed to the inside of the oral cavity 6 with the car 25 travelling on it can be furthermore seen in the drawing.

FIG. 2 shows a detail of the automatic apparatus for tooth-stump preparation introduced in FIG. 1 situated in the oral cavity 6 in top view. The guide rail 24 of horse-shoe shape with the car 25 travelling on it, respectively the combined laser head 26 movable towards all directions situated in the car 25 can be as well seen in the drawing.

FIG. 3 shows the combined laser head 26 in details. The parts of the combined laser head 26 are the laser miller 27, the control optics 28 situated concentrically around it, and the cooling openings 29 situated symmetrically outside the control optics 28 preferably four cooling openings 29.

In case of a definite preferable realization of the solution according to the invention as a first step the dentist makes a 3D X-ray recording of that part of the patient's jaw, where the tooth should be restored with the help of the traditional bridge-technology with the pillar teeth bordering the missing teeth.

The advantage of the X-ray recording is, that it shows the possible hidden problems as well. As an alternative to the X-ray recording, this step can be taken by scanning as an alternative, however by scanning only the surface of the teeth can be visualized, so this method is not suitable for revealing the hidden problems in the depth of the tooth. It is preferable to remove the metal dentures or amalgame fillings from the concerned area before the X-ray recording, because these might disturb producing a clear picture. The metallic pieces on the inert areas obviously can remain in the mouth.

As a second step the dentist digitally designs the structure, shape of the prosthesis to be made with a designing program made for this purpose, respectively designs the form of the stump(s) belonging to the prosthesis to be made, that is designs the preparation of the stump(s). Resulting from this activity a digital design is produced for the dentist.

As a third step, on basis of the ready digital plan the prosthesis is made in the prosthetic dentistry with the known CAD/CAM technology with great accuracy, in given case with hundredth millimeter accuracy.

As a fourth step, also on basis of the ready digital plan, the dentist, or on basis of the dentist's instruction a highly qualified professionally skilled person carries out the preparation of the tooth-stump(s) serving as pillar tooth/teeth. The detailed description of this step is as follows:

1. The dentist with the help of a frame of maxillo-mandibular fixing 14—according to the invention, being part of the apparatus for the automation of dental stump-preparation with laser head, described in FIG. 1 in details modified for the automation of dental stump-preparation,—makes the patient's mandible open to the maximum position and fixes it there. The doctor fixes first the modified maxillo-mandibular area 15, the upper part of the frame of maxillo-mandibular fixing 14, namely the part from the nose bridge 3 to the oral caivty 6 to the maxillo-mandibular area of the patient, then the lower part of the frame of maxillo-mandibular fixing 14 stretching from the oral cavity 6 to the lower part of the jaw 10 is fixed to the maxillo-mandibular area of the patient.

The supporting, fixing points of the frame of maxillo-mandibular fixing 14 are as follows:
nose bridge 3—with the help of cushion 16
access of external acoustic meatus 5 of the two ears 4—with the help of a metal or plastic ball 17
palate 7—with the help of a rubbered palate support 19 of ball shape,
area of retromolar gap 12 on the lower jaw on both sides—with the help of a retromolar support (pad) 18 each,
the part of the jaw tip 11 bending back—with the help of the vacuum pad for fixing jaw tip 20 with polystyrene balls, bearing below the jaw tip 11.

The supporting points of the frame of maxillo-mandibular fixing 14 are connected with each other by telescopic, stiffening studs of pneumatic drive, such a way a uniform structure of the frame of maxillo-mandibular fixing 14 is created. Pneumatics ensures, that the frame of maxillo-mandibular fixing 14 can be adjusted in conformity with the measurements of the given patient. Application of pneumatics is beneficial also, because it can be easily connected with units currently used in dentistries.

Each part of the frame of maxillo-mandibular fixing 14 is made of materials, metal, plastic, or rubbered, or siliconized metal or plastic, that can be easily cleaned, desinfected.

The fixed, open position of the oral cavity ensured by the frame of maxillo-mandibular fixing 14 is especially important when the laser miller 27 works on the lower set of teeth 8, because the lower jaw can move around a rotation axis. This fixed, open position of the mouth is also beneficial, as the laser miller 27 can work on the upper toothrow 9 much more precisely.

Spanning of the mouth is also necessary to provide enough space for the combined laser head 26 travelling on the guide rail 24 making the preparation of the pillar tooth/teeth according to the invention.

The guide rail 24 of horse-shoe shape, following the arc of teeth, fixed to the frame of maxillo-mandibular fixing 14 is a very important part of the automatic tooth-stump preparing apparatus, as the combined laser head 26 situated in the car 25 functioning as carrier can change its place.

The guide rail 24 of horse-shoe shape itself can be also moved vertically downwards and upwards, depending on the place of the pillar tooth to be prepared: in case the pillar tooth to be prepared is located in the lower toothrow 8, then the guide rail 24 must be lowered, in case the pillar tooth to be prepared is located in the upper toothrow 9, then the guide rail 24 must be raised.

The position of the combined laser head 26 situated in the car 25 on the guide rail 24, also depends on the location of the pillar tooth that is whether the pillar thooth is situated in the lower toothrow 8 or situated in th upper toothrow 9:

in case the pillar tooth to be prepared is located in the lower toothrow 8, then the combined laser head 26 situated in the car 25 is located on the lower part of the guide rail 24, in case the pillar tooth to be prepared is located in the upper toothrow 9, then combined laser head 26 in the car 25 is situated on the upper part of the guide rail 24.

2. The computer directs the combined laser head 26 in the car 25 to the pillar tooth to be actually prepared on basis of the digital plan. The combined laser head 26 in the car 25 can move to every direction of the space along the hemisphere shell.

The mobility of the combined laser head 26 to every direction of the space can be solved in such a way, that the combined laser head 26 in the car 25 is positioned on a lever, and the computer directs the movement of the lever, or it can be solved in such a way as well, that the combined laser head 26 in the car 25 is located on a hemisphere shell, and the computer directs the movement of the combined laser head 26 on the hemisphere shell.

3. The combined laser head 26 directed to the right place can carry out the preparation of the pillar tooth/teeth on basis of the ready digital plan, controlled digitally by the computer, with the supervision of the dentist, or a professionally skilled person, this way faults resulting from the inaccuracy of trembling of human hands can be eliminated.

As can be seen in FIG. 3, the combined laser head 26 includes the laser miller 27, the control optics 28, and the cooling openings 29. The laser miller 27 emitting hard laser beams carries out directly the preparation of the pillar tooth/teeth on basis of the ready digital plan, controlled by the computer with great accuracy, in given case with hundredth millimeter precisity. The control optics 28 surrounding concentrically the laser miller 27 is actually a “digital eye” sending signals to the control computer by emitting soft laser beams, scanning the surroundings on the stage of the preparation, and compares the actual stage of preparation with the original plan, and notices the smallest deviations. The principle of the functioning is the digital frame, rendered to the physical frame fixed at the physical reference points. During preparation the control optics 28 search for the marked reference points in the digital frame—designated during the computer planning and checks the laser miller 27 accordingly.

The laser miller 27 and the control optics 28 function either parallel with each other continously, or the laser miller 27 and the control optics 28 function alternating within short periods. The continous, or frequent control of the process of milling is very important, because if the laser miller 27 mills on the wrong place resulting from any deficiency of the system, then it is very important, that the computer is “informed” about it within the shortest possible time, and “stop” or correct the laser miller 27 carrying out the preparation, excluding this way for example the faults resulting from the possible displacement.

The control optics 28 surrounding concentrically the laser miller 27 are symmetrically surrounded by the cooling openings 29—preferably four pieces of cooling openings 29—

on the combined laser head 26. During milling the cooling water from the cooling openings 29 cools steadily down the tooth under preparation. It is needed, because resulting from laser milling heat is developed, which can damage the pulp, therefore cooling is required.

The following complements join the combined laser head 26:
glass fibre leading the hard laser beam from the laser generator to the laser miller 27,
another glass fibre leading the soft laser beam carrying out the control in the control optics 28 to the computer,
tubes, preferably four pieces of tubes, leading the cooling water to the cooling openings 29.

It is true for every part of the apparatus according to the invention, located in the mouth, that they should be produced in the possible smallest size, as ab ovo the room is limited in the oral cavity 6, respectively it is also more beneficial for the patient if a small device “is working” in their mouth.

The following complementary devices are necessary for the proper functioning,—convenient for the patient as well—of the apparatus according to the invention:
A silicon-rubber sheet usually used in dentistry, separating safely the oral cavity 6 from the pharynx 13 preventing the cooling water getting to the pharynx 13. This silicon-rubber sheet is fixed to the proper parts of the frame of maxillo-mandibular fixing 14 in such a way, that with the help of the parts situated in the mouth of the modified maxillo-mandibular area 15 the silicon-rubber sheet is strained—at two points at the retromolar gap 12, in the middle of the palate 7, and at two points at the tuber maxilae on the upper jaw.
the water suction tube (saliva pump) usual in dentistry ensures the removal of the cooling water and the saliva.

As step five the dentist places the prothetical piece—bridge, crown, insert, etc.—produced by CAD/CAM technique with great accuracy, in given case with hundredth millimeter precision according to the general practice,—into the mouth, resulting from the great exactness without subsequent adjusting, and fixes them on to the prepared pillar tooth/teeth.

This case the patient has to have only one appointment with the dentist. 3D X-ray recording can be made anywhere in the world, and it can be forwarded even by e-mail to the dentist, respectively to the prosthetic dentistry. On basis of the ready X-ray recording the digital plan can be made, on the basis of which the prosthesis is made in the prosthetic dentistry. The patient goes to see the dentist only afterwards, who prepares the tooth-stump, also on basis of the ready digital plan. As a last step also this case the dentist places the ready prosthesis into the mouth on the prepared tooth-stump according to the usual practice.

During the process it is preferable to keep the patient in completely horizontal state, as the supporting device located on different maxillo-mandibular areas is the most convenient to wear in this position.

In case of another preferable definite realization of the solution according to the invention also the abovementioned procedure is realized, with one difference, that the order of the step three and four is exchanged. Namely, on basis of the ready digital plan the dentist first makes the preparation of the tooth-stumps serving as pillar tooth/teeth, then also on basis of the ready digital plan the prosthesis is made in the prosthetic dentistry with the known CAD/CAM technology.

The benefit of the solution according to the invention is, that subjective factors can be eliminated resulting from the fixing of the tooth-stump preparing apparatus on different maxillo-mandibular areas, respectively precise, in given case hundredth millimeter accuracy tooth-stump preparation can be realized even under difficult anatomical conditions, and a tired, perhaps unfit dentist of average manual skills is also able to make tooth-stump preparation conforming with high level quality requirements.

Another benefit of the solution according to the invention is, that the patient needs to go to the dentist once only, this way dental treatment accelerates revolutionarily, as following the preparation of the tooth-stump it is not needed to wait for days, or even weeks to realize the work in the prosthetic dentistry.

A further advantage of the solution according to the invention is, that no impression-taking is needed, which is partly an absolutely unpleasant experience for a lot of patients, partly a significant source of faults, partly expensive materials of great quantity—material for impression taking, laboratory chemicals, etc.—can be saved by it.

An additional benefit of the solution according to the invention is, that no mushbite registration is needed, resulting also in high discrepancy percentage.

The advantage of the 3D X-ray picture used for scanning during the solution according to the invention is, that it can show not only the surface of teeth, but also the possible hidden problems as well.

The advantage of the laser tooth-stump preparation applied during the solution according to the invention is, that the tooth, and by this the patient as well endures better working with laser, than the traditional cutting, due to the fact, that laser causes micro-discharges and the treatment is painless for the patient, if the safety regulations are kept. A further virtue of the apparatus according to the invention is, that with the help of the multiflex clutch it can be easily joined the currently used dental unit-apparatus, the cooling water tube can be joined the exhaustor of the unit and pneumatics are also easily connected to the uniform devices used presently in dentistries.

Another benefit of the solution according to the invention is, that after having made the digital plan and fixed the apparatus carrying out the preparation on the facial area of the patient, no active participation from the dentist is required, only a professionally highly skilled person is needed, who can stop the machine in case of a possible failure and removes it from the mouth. So during this time the dentist can deal with another patient, instead of wasting his time with lengthy tooth-stump preparation, taking impressions and making temporary dentures, increasing the efficiency of the dentist to a great extent. All in all by decreasing the number of appointments with the dentist, respectively the time of one patient's treatment, the patient turnover can be increased and much more treatment can be given in a given time period.

Also there is a further advantage for the patient, that the treatment is quite bearable, respectively that patients like to choose dentists applying state of the art technology.

### LIST OF REFERENCES

1—maxillo-mandibular area

2—nose

3—nose ridge

4—ear

5—access of external acoustic meatus (two pcs)

6—oral cavity

7—palate

8—lower toothrow

9—upper toothrow

10—jaw

11—jaw tip

12—retromolar gap (two pcs)

13—pharynx

14—frame of maxillo-mandibular fixing

15—modified maxillo-mandibular area

16—cushion

17—ball

18—retromolar support (pad)

19—palate support

20—vacuum pad for fixing jaw tip

21—stud for sustaining jaw (two pcs)

22—stud for supporting palate

23—stud for fixing jaw tip

24—guide rail

25—car

26—combined laser head

27—laser miller

28—control optics

29—cooling openings

1. Process for the automation of dental stump preparation, during which process the designing of the preparation of the dental stump(s) serving as pillar tooth/teeth for the fixing of the prothetic piece—bridge, crown, insert,—to be placed into the oral cavity takes place by the dentist on basis of a three-dimensional X-ray recording, taken from the relative spot of the jaw, on computer with the help of a designing-program made for this purpose, then, on basis of the complete digital plan, producing the prosthesis takes place with CAD/CAM technology, then on basis of the same digital plan, preparation of the dental stump(s) serving as pillar tooth/teeth takes place also by the dentist with laser head dental milling device,
characterized by that,
during the preparation of the dental stump(s)
as Step 1 the dentist, or a professionally qualified person persuades the patient's mandible into maximum opening position with the help of the telescopic frame of maxillo-mandibular fixing (14) and fixes it there,
as Step 2 on basis of the complete digital plan the computer directs the combined laser head (26) in the car (25) to the pillar tooth to be prepared,
as Step 3 the laser miller (27) with mobility towards every direction of the space situated on the combined laser head (26) directed to the proper spot, carries out the preparation of the pillar tooth on basis of the complete digital plan, digitally controlled by the computer, under the supervision of the dentist, or a professionally qualified person, with the supervision of the control optics (28).

2. Process according to claim 1 characterized by that, that at first the upper part of the frame of maxillo-mandibular fixing (14), from the nose bridge (3) to the oral cavity (6) is fixed to the patient's maxillo-mandibular area, then, following this the lower part of the frame of maxillo-mandibular fixing (14), the part from the oral cavity (6) to the bottom of the jaw (10) is fixed to the patient's maxillo-mandibular area with the help of the pneumatically functioning telescopic formation of the frame of maxillo-mandibular fixing (14) conforming with the measurements of the given patient.

3. Process according to claim 1 characterized by that, during preparation the control optics (28) automatically finds the reference points in the digital frame, marked during the computer designing, and comparing with them, controls the laser miller (27), so the combined laser head (26) is directed by the computer on the complete digital plan in such a way, that it compares the digital frame with the signals sent by the control optics (28).

4. Process according to claim 1 characterized by that, the laser miller (27) and the control optics (28) operate continuously, parallel with each other.

5. Process according to claim 1 characterized by that, the laser miller (27) and the control optics (28) operate alternating, within short, subsequent periods.

6. Process according to claim 1 characterized by that, during milling the cooling water coming from the cooling openings (29) cools down continuously the actual tooth under preparation.

7. Apparatus for the automation of dental stump preparation, the said apparatus is provided with control optics for surveying the preparation of tooth stump(s), which is made by a combined laser head containing a laser miller emitting hard laser beam,
characterized by that,
the supporting, fixing points of the telescopic frame of maxillo-mandibular fixing (14) on the patient's maxillo mandibular area (1) comprising part of the automatic tooth stump preparing apparatus, are as follows:
the nose ridge (3)—with the help of the cushion (16),
the acces of external acoustic meatus (5) of both ears (4)—with the help of a metallic or plastic ball (17) each,
the palate (7)—with the help of the rubbered palate support (19) of ball shape,
the area of retromolar gap (12) situated on both mandibles—with the help of the retromolar support (pad) (18),
the part of the jaw tip (11) bending back,—with the help of the vacuum pad for fixing jaw tip (20) with polystyrene balls, bearing below the jaw tip (11),
the combined laser head (26) carrying out the milling travells in the car (25) placed on the horse-shoe-shaped guide rail (24) following the arc of the teeth, fixed on the frame of maxillo-mandibular fixing (14) of the automatic tooth stump preparing apparatus.

8. Apparatus according to claim 7 characterized by that, the supporting points of the frame of maxillo-mandibular fixing (14), are connected with each other by a telescopic stiffening stud, movable pneumatically, this way the complete frame of maxillo-mandibular fixing (14) is a uniform structure.

9. Apparatus according to claim 7 characterized by that, each part of the frame of maxillo-mandibular fixing (14), is made of materials of easy cleansing and disinfecting, preferably from metal, plastic, or rubberized, or siliconized metal or plastic.

10. Apparatus according to claim 7 characterized by that, in case the pillar tooth to be prepared is in the upper toothrow (9), then the combined laser head (26) situated in the car (25) is on the upper part of the guide rail (24), and the guide rail (24) of horse-shoe shape itself is set near the upper toothrow (9).

11. Apparatus according to claim 7 characterized by that, in case the pillar tooth to be prepared is in the lower toothrow (8), then the combined laser head (26) in the car (25) is situated on the lower part of the guide rail (24), and the guide rail (24) of horse-shoe shape itself is located near the lower toothrow (8).

12. Apparatus according to claim 7 characterized by that, the control optics (28) situated concentrically around the laser miller (27) on the combined laser head (26) and the cooling openings (29) preferably four pieces of cooling openings (29) are situated symmetrically outside the control optics (28).

13. Apparatus according to claim 7 characterized by that, the laser miller (27) is preferably erbium laser miller (27).

14. Apparatus according to claim 7 characterized by that, the control optics (28) of the combined laser head (26) is preferably a laser control optics (28) emitting soft laser beams.

15. Apparatus according to claim 7 characterized by that, the combined laser head (26) in the car (25) is positioned on a lever, and the computer controlling the preparation directs the movement of the lever, so the combined laser head (26) is able to move along the hemisphere shell.

16. Apparatus according to claim 7 characterized by that, the combined laser head (26) in the car (25), able to move along the hemisphere shell is located on the shell of a hemisphere, and the movement of the combined laser head (26) on the shell of the hemisphere is directed by the computer controlling the preparation.

17. Apparatus according to claim 7 characterized by that, the following complements join the combined laser head (26):
glass fibre leading the hard laser beam from the laser generator to the laser miller (27),
another glass fibre leading the soft laser beam carrying out the control in the control optics (28) to the computer controlling the milling,
tubes, preferably four pieces of tubes, leading the cooling water to the cooling openings (29).

18. Apparatus according to claim 7
characterized by that, additional supplementary devices of the automatic tooth stump preparing apparatus are as follows:
a silicon-rubber sheet usually used in dentistry, separating safely the oral cavity (6) from the pharynx (13), fixed to the proper parts of the frame of maxillo-mandibular fixing (14) at five points, and these five points are the following:
two points at the retromolar gap (12),
one point in the middle of the palate (7)
two points at the tuber maxillae on the upper jaw,
as well as a water suction tube (saliva pump) generally used in dentistry ensuring the removal of the cooling water and the saliva in a proper way.
